(19) United States
(12) Patent Application Publication
(10) Pub. No.: US 2019/0083206 A1
(43) Pub. Date: Mar. 21, 2019

(54) DENTAL IMPLANT METHOD AND APPARATUS

(71) Applicants: Josef F. Visinski, Seymour, CT (US); Andrew Visinski, Seymour, CT (US)

(72) Inventors: Josef F. Visinski, Seymour, CT (US); Andrew Visinski, Seymour, CT (US)

(21) Appl. No.: 16/195,964

(22) Filed: Nov. 20, 2018

Related U.S. Application Data
(60) Provisional application No. 62/588,958, filed on Nov. 21, 2017.

(51) Int. Cl. A61C 8/00; A61C 9/00; A61C 13/00; A61B 5/055; A61B 6/03; A61B 6/14

(52) U.S. Cl. CPC A61C 8/0036; A61C 9/0046; A61C 13/0004; A61B 5/055; A61C 2008/0046; A61B 6/14; A61B 6/037; A61C 8/0043; A61B 6/032

(57) ABSTRACT

A method of manufacturing a dental implant wherein the resulting dental implant closely corresponds to or matches the size and shape of the extracted tooth. Generally stated, the method comprises the steps of (i) three dimensionally mapping the size and shape of the crown and root of a tooth to be extracted and (ii) using said mapping to manufacture a dental implant to correspond to the size and the shape of said crown and root of the tooth to be extracted. Further, the resulting dental implant may comprise a number of anti-migratory structural features that function to better secure the dental implant to the patient.

### CROSS-REFERENCE TO RELATED APPLICATION

This application claims priority from Provisional Patent Application Ser. No. 62/588,958 filed on Nov. 21, 2017.

### FIELD OF THE INVENTION

This invention relates to a method of manufacturing a dental implant that is substantially similar (if not exact) in size and shape to the tooth that it is replacing.

### BACKGROUND

Oftentimes, it is necessary to pull or extract a tooth from a dental patient's (human or animal) mouth. Generally stated, a tooth is comprised of a crown portion and a root portion, which may be comprised of one or more roots. The crown portion extends outwardly from the patient's gums, and is exposed to the interior of the patient's mouth. By comparison, the root portion and its various roots extend into the gums of the patient and serves as an anchor for the tooth to the jawbone. More specifically, the root is attached to the tooth-bearing bone of the jaw by a fibrous ligament, which is oftentimes referred to as the periodontal ligament or membrane.

When a tooth is pulled, the void is oftentimes replaced with a denture or dental implant. Further, the manufacturing of dentures and dental implants is oftentimes performed based upon the approximate size of the tooth being extracted, with more focus being placed on the crown portion of the tooth to ensure that no unsightly gaps between the dental implant and the adjacent teeth are created. Additionally, as described and shown below, the root portion of the dental implant is oftentimes in the general form of a screw. Unfortunately, the size and shape of the root or screw does not correspond to the opening or void in the gums created by the pulling of the extracted tooth. Because this is true, the dental patient will typically experience more pain during the dental implant procedure, and the recovery period will be longer.

Consequently, there is a long felt need in the art for a method of manufacturing a dental implant wherein the resulting dental implant corresponds or matches the size and shape of the extracted tooth (i.e., the crown portion, the root portion, or both). There is also a long felt need in the art for a dental implant having an anchoring system that allows for the secure attachment of the dental implant to the patient's jawbone. Finally, there is a long felt need for a dental implant device that accomplishes all of the forgoing objectives, and that is relatively inexpensive to manufacture and safe and easy to use.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed herein, in one aspect thereof, is a method of manufacturing a dental implant wherein the resulting dental implant corresponds or matches the size and shape of the tooth to be extracted (i.e., the crown portion, the root portion, or both). Generally stated, the method comprises the steps of (i) three dimensionally mapping the size and shape of the crown and root of a tooth to be extracted and (ii) using said mapping to manufacture a dental implant to correspond to the size and the shape of at least one of said crown and root of the tooth to be extracted. The method and resultant dental implant can be used with both metallic and non-metallic implants, and can be used in both humans and animals.

In another embodiment of the present invention, the root portion of the resulting dental implant will further comprise one or more anchoring mechanisms that provide for the secure attachment of the dental implant to the patient's jawbone. The anchoring mechanisms can be in the form or groves, steps or the like, or may further comprise an opening for receipt of a fastener that could be used to secure the dental implant to the jawbone.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 is a perspective view of a prior art dental implant installed in a patient's mouth.

FIG. 2 is a perspective view of an extracted tooth.

FIG. 3 is a perspective view of a dental implant manufactured by the method of the present invention to correspond to the size and shape of the extracted tooth shown in FIG. 2.

FIG. 4 is a flowchart generally stating the method steps of the present invention.

FIG. 5 is a perspective view of an alternative embodiment of the dental implant manufactured by the method of the present invention.

FIG. 6 is a perspective view of an additional alternative embodiment of the dental implant manufactured by the method of the present invention.

FIG. 7 is a perspective view of an additional alternative embodiment of the dental implant manufactured by the method of the present invention.

FIG. 8 is a perspective view of an additional alternative embodiment of the dental implant manufactured by the method of the present invention.

FIG. 9 is a perspective view of an additional alternative embodiment of the dental implant manufactured by the method of the present invention.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details.

Generally stated, the method of manufacturing a dental implant of the present invention permits the resulting dental implant to correspond to or match the size and shape of the extracted tooth (i.e., the crown portion, the root portion, or both), and may further comprise one or more anchoring systems that allow for the secure attachment of the dental implant to the patient's jawbone. The method and resultant dental implant can be used with both metallic and non-metallic dental implants, and can be used in both humans and animals.

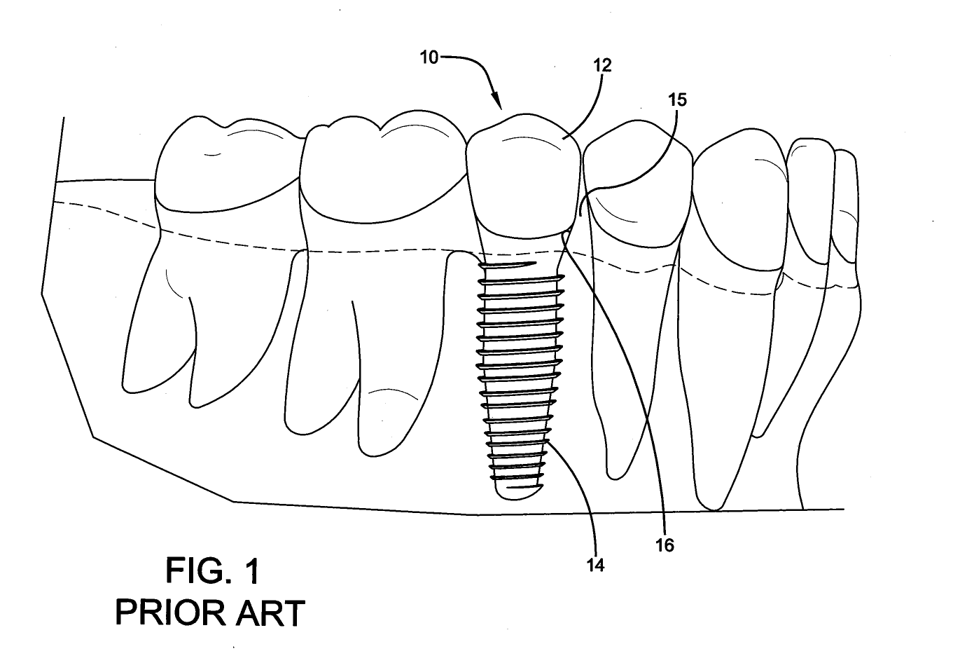

Referring initially to the drawings, FIG. 1 is a perspective view of a prior art dental implant 10 installed in a patient's mouth. Prior art dental implant 10 is comprised of a crown 12 and a root 14, wherein root 14 is typically in the configuration of a screw. Crown 12 extends outwardly from a patient's gums 15, and root 14 is inserted into an opening 16 in gums 15, as best shown in FIG. 1, and secures to the patient's jawbone. However, as previously stated, because opening 16 in the gum 15 does not precisely correspond to the size and shape of the root 14, the patient will experience more pain during the procedure and the recovery period will be longer as the gum and jawbone will need to eventually grow to fill any voids left by the pulled tooth that are not filled by prior art implant 10. Unfortunately, the void could also be a haven for food and/or bacteria to accumulate, which could result in bone loss, adjacent tooth decay, infections and/or general discomfort for the patient. The unique method or manufacture and resulting dental implant of the present invention alleviates these issues and results in a speedier and less painful recovery for the patient.

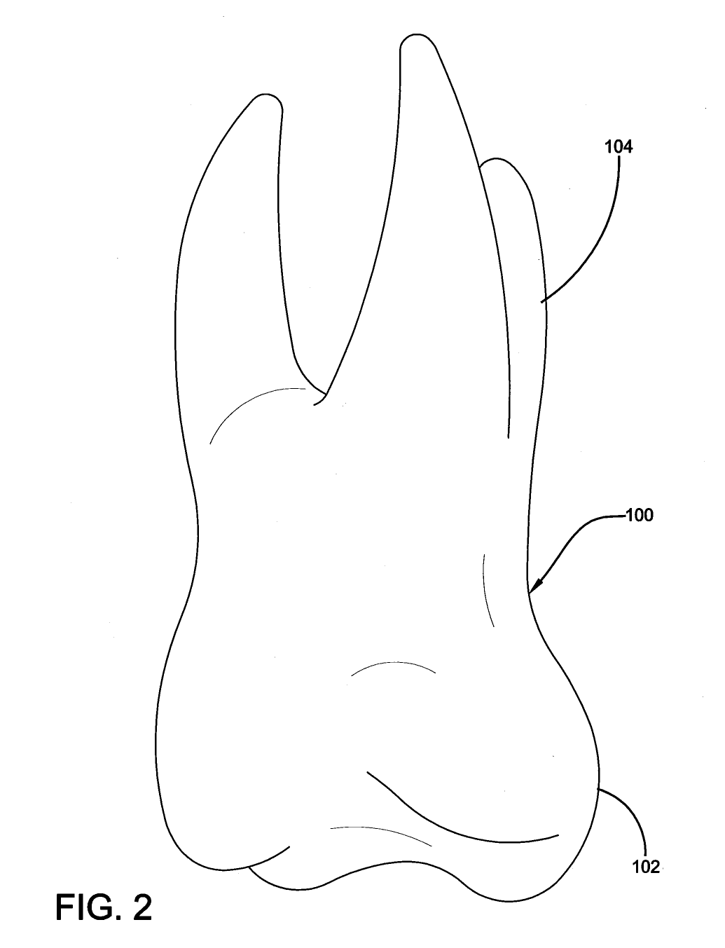

FIG. 2 is a perspective view of an extracted tooth 100. Extracted tooth 100 is comprised of a crown section 102 and a root section 104. As previously mentioned, the crown and root sections 102, 104 of tooth 100 are unique in both size and shape, and it is extraordinarily unlikely that any two teeth would have the exact same size and shape of crown and/or root.

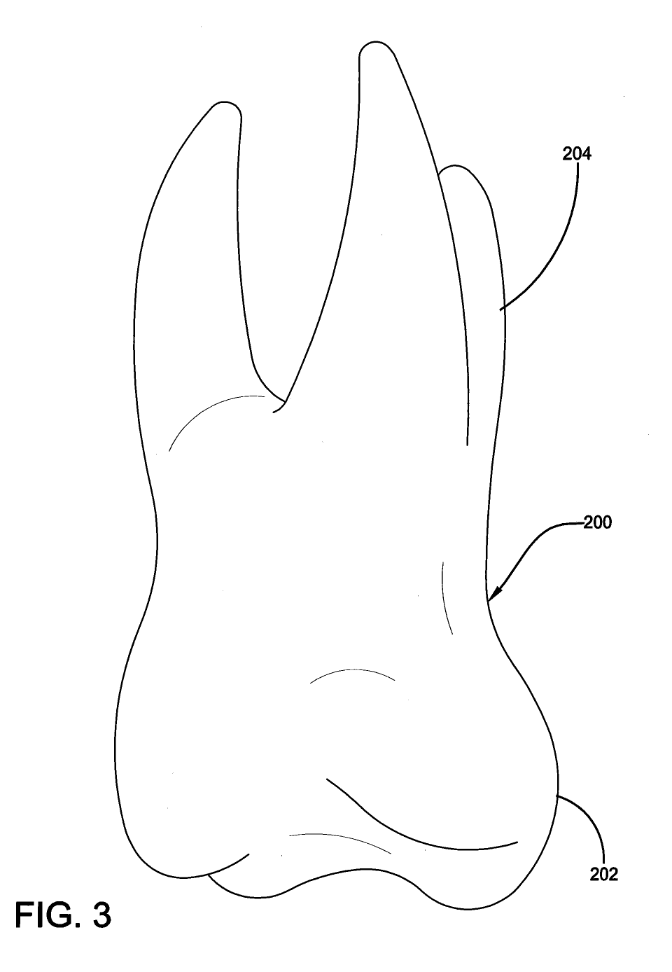

FIG. 3 is a perspective view of a dental implant 200 manufactured by the method of the present invention to correspond to the size and shape of the extracted tooth shown in FIG. 2. Dental implant 200 is preferably comprised of a crown portion 202 and a root portion 204. Crown portion 202 and root portion 204 are preferably integrally formed, but are not required to be. In a preferred embodiment of the present invention, the size and shape of root portion 204 corresponds to the size and shape of root section 104 of extracted tooth 100. In a further preferred embodiment of the present invention, the size and shape of crown portion 202 corresponds to the size and shape of tooth section 102 of extracted tooth 100, though the same is not required. For example, it may be desirable to manufacture a crown portion 220 that does not contain any of the structural defects or deficiencies of the crown section 102 of extracted tooth 100. Nonetheless, in a most preferred embodiment of the present invention, the size and shape of crown portion 202 and root portion 204 of dental implant 200 are substantially similar, if not identical, to crown section 102 and root section 104 of extracted tooth 100.

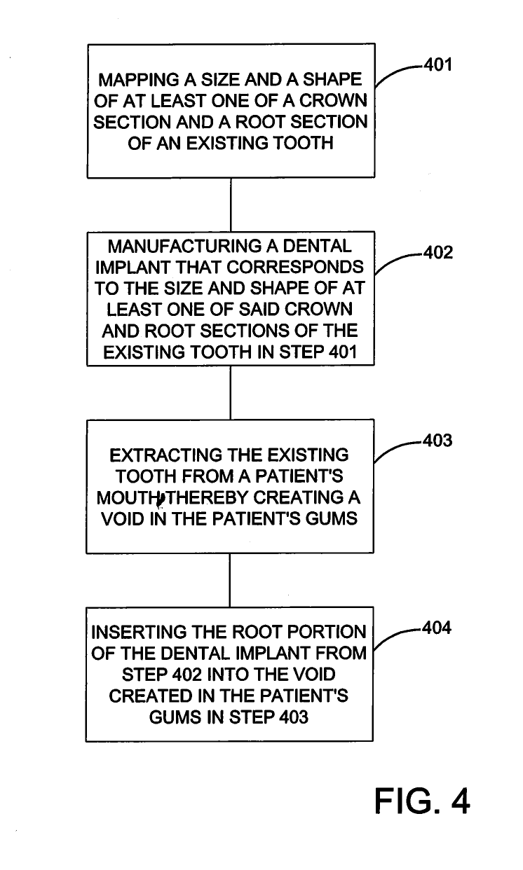

FIG. 4 is a flowchart generally stating the method steps of the present invention. More specifically, block 401 describes the initial step in the method of the present invention, namely three dimensionally mapping the size and/or shape of at least one of the crown section 102 and the root section 104 of the tooth 100 to be extracted. In a preferred embodiment of the present invention, the three dimensional mapping is performed while the tooth 100 is still in the patient's mouth, and can be performed using any existing technology known in the art for three dimensionally mapping an object including, without limitation, magnetic resonance imaging, x-ray computed tomography, positron emission tomography, etc.

Following the successful mapping of the tooth 100 to be extracted in step 401, the next step 402 is to use said mapping results to manufacture a dental implant 200 by any common means of three dimensional manufacturing known in the art, wherein at least one of the size or shape of dental implant 200 corresponds to the size and/or shape of extracted tooth 100. Said three dimensional manufacturing methods may include, but are not limited to, molding, additive manufacturing, forming, etc. As previously stated, ideally, the entire dental implant 200 would correspond in size and shape to extracted tooth 100. Once dental implant 200 has been manufactured, the method of the present invention further comprises the step 403 of extracting tooth 100 from the patient's mouth to create a void or opening 16 in the patient's gums 15. Once said opening 16 has been created, at step 404, the root portion 204 of dental implant 200 may be inserted into said void opening. Further, because the size and shape of dental implant 200 substantially (if not identically) matches the size and shape of extracted tooth 100, root portion 204 of dental implant 200 should fit snugly into opening 16. It will be appreciated by those of ordinary skill in the art that the method of manufacture of the present invention and the resultant dental implant 200 can be used with both metallic and non-metallic implants, and can also be used in both humans and animals.

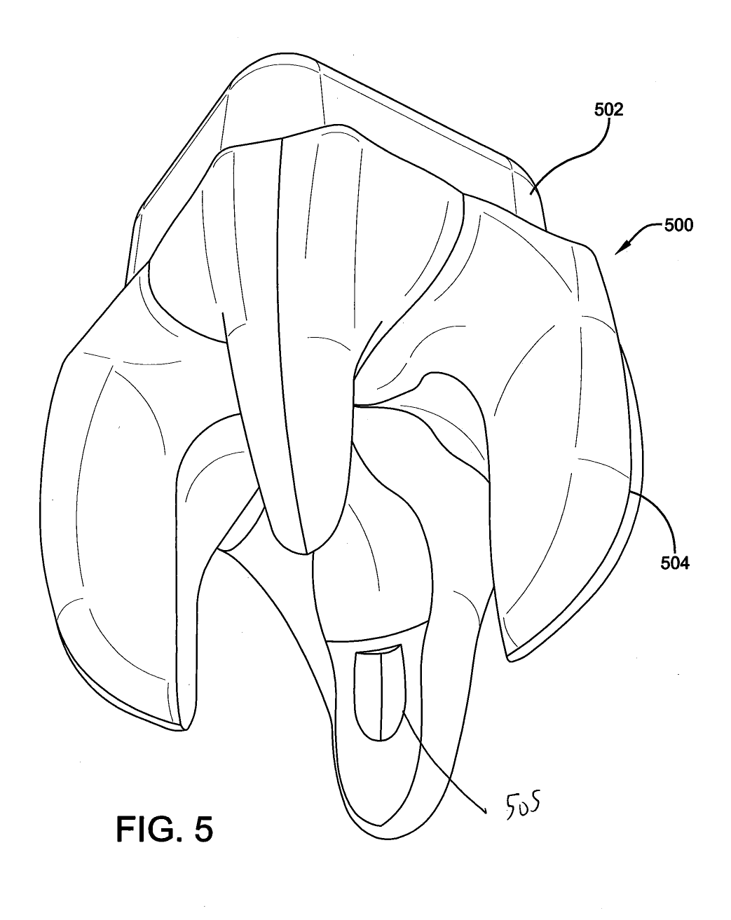

FIG. 5 is a perspective view of an alternative embodiment of a dental implant 500 manufactured by the method of the present invention. Dental implant 500 is comprised of a crown section 502 and a root section 504. Crown section 502 and root section 504 are preferably integrally formed, but are not required to be. In this particular embodiment, portions 505 of root section 504 may be undercut to allow for a better fit in the patient's jaw bone (not shown). It is also contemplated that bone graft materials (not shown) can be used to fill in any remaining openings between the jaw bone and dental implant 500 for a better fit. Said bone graft material can be, but is not limited to, an autograft, allograft, xenograft, alloplast, homograft, etc.

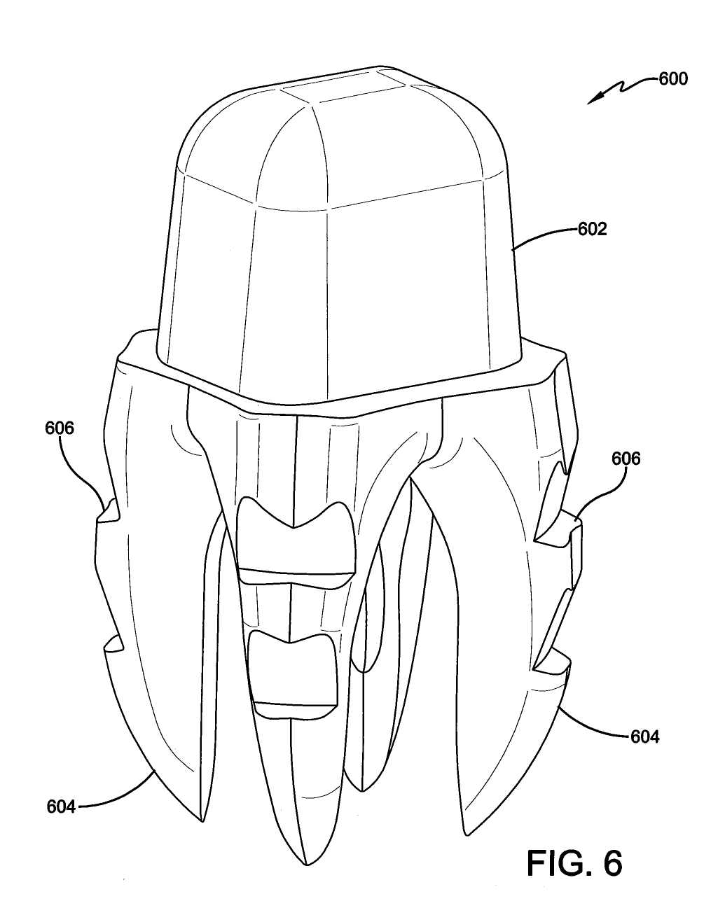

FIG. 6 is a perspective view of a further alternative embodiment of a dental implant 600 manufactured by the method of the present invention. Dental implant 600 is preferably comprised of a crown section 602 and a root section 604. Crown section 602 and root section 604 are preferably integrally formed, but are not required to be. In this particular embodiment, root sections 604 may further comprise anti-migratory anchoring grooves or steps 606 that further anchor dental implant 600 to the patient's jaw bone (not shown). Similar to the above referenced embodiments, bone graft materials (not shown) can be used to fill in any remaining openings between the jaw bone and dental implant 600 for a better fit. Said bone graft material can be, but is not limited to, an autograft, allograft, xenograft, alloplast, homograft, etc.

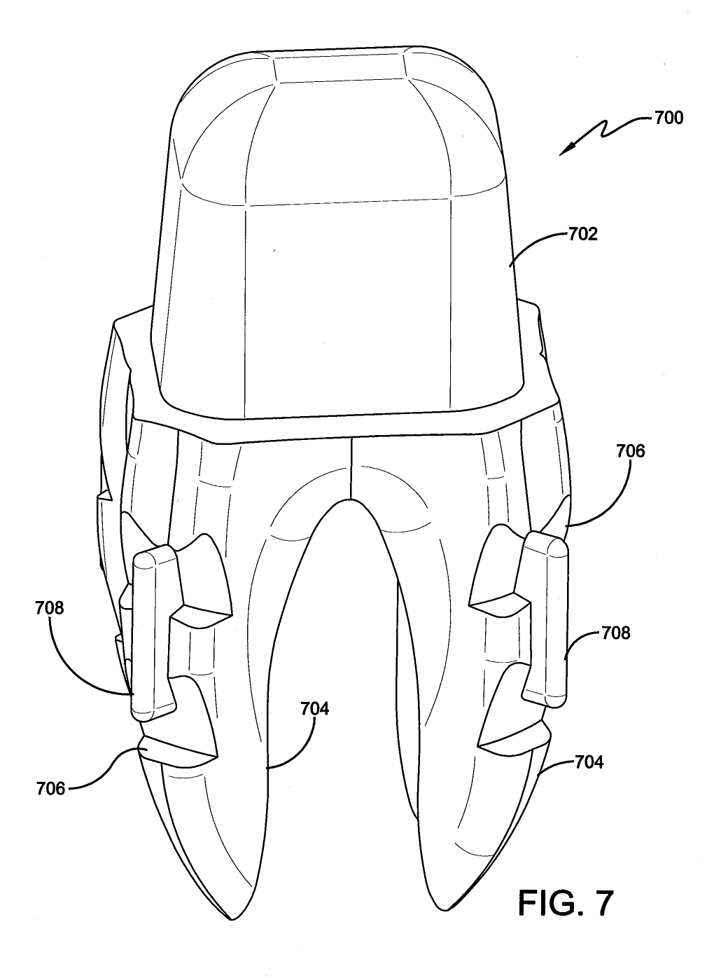

FIG. 7 is a perspective view of yet another alternative embodiment of dental implant 700 that can be manufactured by the method of the present invention. Dental implant 700 is comprised of a crown section 702 and a root section 704. Crown section 702 and root section 704 are preferably integrally formed, but are not required to be. In this particular embodiment, root sections 704 may further comprise anti-migratory anchoring grooves or steps 706 and/or protrusions 708 that further anchor dental implant 700 to the patient's jaw bone (not shown). Similar to the above referenced embodiments, bone graft materials (not shown) can be used to fill in any remaining openings between the jaw bone and dental implant 700 for a better fit. Said bone graft material can be, but is not limited to, an autograft, allograft, xenograft, alloplast, homograft, etc.

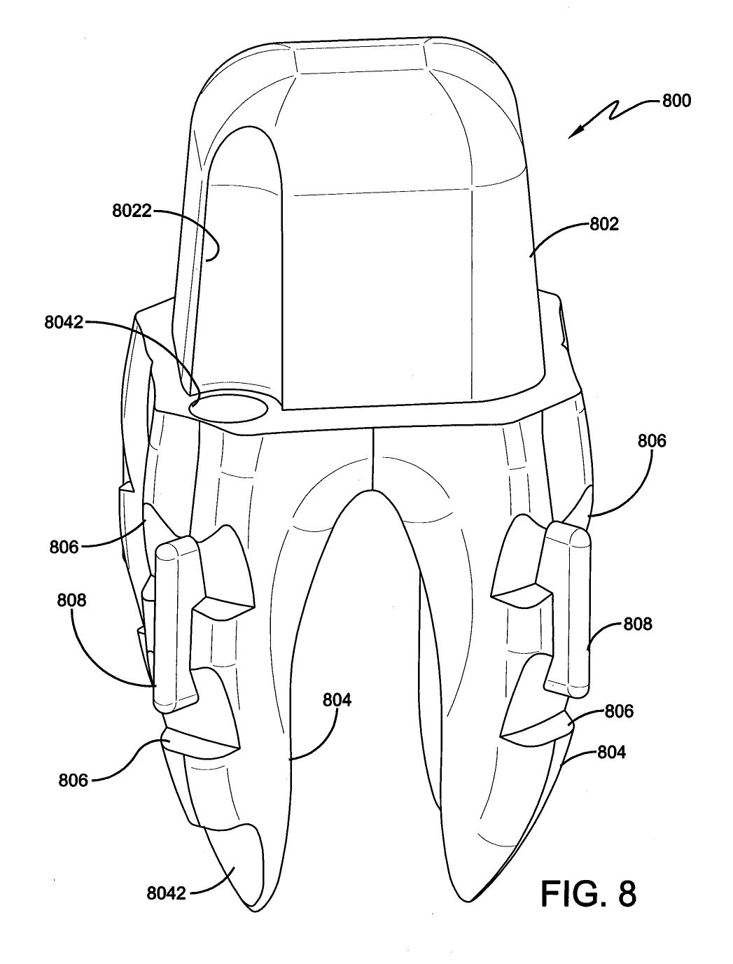

FIG. 8 is a perspective view of yet another alternative embodiment of dental implant 800 manufactured in accordance with method of the present invention. Dental implant 800 is comprised of a crown section 802 and a root section 804. Crown section 802 and root section 804 are preferably integrally formed, but are not required to be. Crown section 802 may further comprise one or more openings or grooves 8022 to accommodate a screw (not shown) that can be used to further attach implant 800 to a patient's jaw (not shown). As best shown in FIG. 8, root sections 804 may further comprise anti-migratory anchoring grooves or steps 806 and protrusions 808 that further anchor dental implant 800 to the patient's jaw bone. Root section 804 may also comprise an opening or groove 8042 therein to accommodate a screw (not shown) that can be used to further attach implant 800 to a patient's jaw.

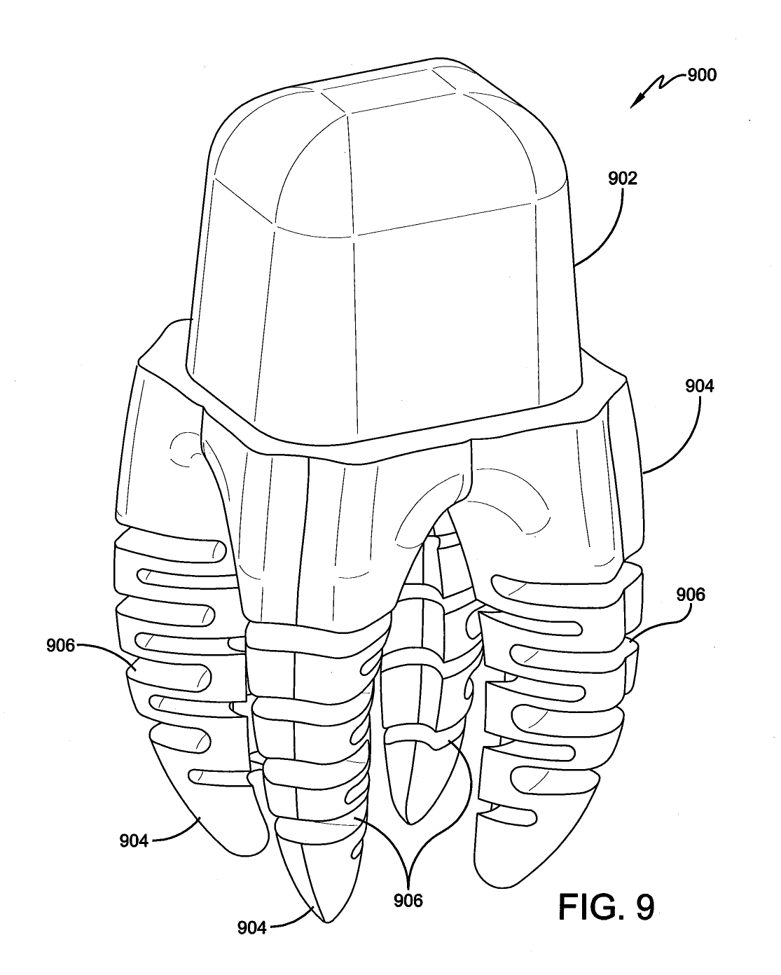

FIG. 9 is a perspective view of an additional alternative embodiment of dental implant 900 that may be manufactured by the method of the present invention. Dental implant 900 is comprised of a crown section 902 and a root section 904. Crown section 892 and root section 904 are preferably integrally formed, but are not required to be. Root sections 904 may further comprise a plurality of openings 906 therein to provide greater flexibility in root sections 904 during the implantation process and to permit osteo-integration between dental implant 900 and the patient's jawbone following implantation. Openings 906 can also later be filled with bone material (not shown).

Clearly, other variations are also within the spirit of the present invention. Thus, while the invention is susceptible to various modifications and alternative constructions, a certain illustrated embodiment thereof is shown in the drawings and has been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of manufacturing a dental implant to replace a tooth comprised of a crown section and a root section comprising the steps of:
mapping a size and a shape of at least one of said crown section and said root section; and
manufacturing the dental implant to correspond to the size and the shape of at least one of said crown section and said root section.

2. The method of claim 1, wherein said mapping is performed by scanning at least one of said crown section and said root section with magnetic resonance imaging.

3. The method of claim 1, wherein said mapping is performed by scanning at least one of said crown section and said root section with x-ray computed tomography.

4. The method of claim 1, wherein said mapping is performed by scanning at least one of said crown section and said root section with positron emission tomography.

5. The method of claim 1 further comprising the steps of extracting the tooth from a patient's mouth, thereby creating a void in a patient's gums and inserting a root portion of said dental implant into said void.

6. A dental implant for replacing an extracted tooth comprising:
a crown portion; and
a root portion, wherein said extracted tooth comprises a crown section and a root section and further wherein said root portion corresponds in shape to said root section.

7. The dental implant of claim 6, wherein said root portion corresponds in size to said root section.

8. The dental implant of claim 6, wherein said crown portion corresponds in shape to said crown section.

9. The dental implant of claim 6, wherein said crown portion corresponds in size to said crown section.

10. The dental implant of claim 6, wherein said root portion further comprises an anchoring groove.

11. The dental implant of claim 6, wherein said root portion further comprises an anchoring groove and a protrusion.

12. The dental implant of claim 6, wherein said crown portion comprises an opening therein for receipt of a fastener, and said root portion further comprises an opening therein for receipt of the fastener.

13. The dental implant of claim 6, wherein said root portion further comprises a plurality of openings therein for increasing the flexibility of said root portion.

14. A method of manufacturing a dental implant to replace a tooth comprised of a crown section and a root section comprising the steps of:
mapping a size and a shape of said crown section and said root section; and
manufacturing the dental implant to correspond to the size and the shape of said crown section and said root section.

15. The method of claim 14, wherein said mapping is performed by scanning said crown section and said root section with magnetic resonance imaging.

16. The method of claim 14, wherein said mapping is performed by scanning said crown section and said root section with x-ray computed tomography.

17. The method of claim 14, wherein said mapping is performed by scanning said crown section and said root section with positron emission tomography.

18. The method of claim 14, wherein the dental implant is comprised of a crown portion and a root portion.

19. The method of claim 18, wherein said root portion further comprises an anchoring groove and a protrusion.

20. The method of claim 18, wherein said crown portion comprises an opening therein for receipt of a fastener, and said root portion further comprises an opening therein for receipt of the fastener.
